(19) United States
(12) Patent Application Publication
(10) Pub. No.: US 2007/0185084 A1
(43) Pub. Date: Aug. 9, 2007

(54) COMPOSITIONS OF AN ANTICONVULSANT AND METHODS OF USING THE SAME FOR REVERSING WEIGHT GAIN

(75) Inventors: Anthony A. McKinney, San Diego, CA (US); Gary Tollefson, Indianapolis, IN (US); Michael A. Cowley, Portland, OR (US)

Correspondence Address: KNOBBE MARTENS OLSON & BEAR LLP, 2040 MAIN STREET, FOURTEENTH FLOOR, IRVINE, CA, 92614, US

(73) Assignee: Orexigen Therapeutics, Inc., San Diego, CA (US)

(21) Appl. No.: 11/622,363

(22) Filed: Jan. 11, 2007

Related U.S. Application Data
(60) Provisional application No. 60/759,116, filed on Jan. 12, 2006.

(51) Int. Cl. A61K 31/551

(52) U.S. Cl. 514/214.02; 514/220; 514/259.41; 514/284; 514/321; 514/359; 514/379; 514/649

(57) ABSTRACT

Disclosed are compositions for reversing weight gain comprising a first compound and a second compound, where the first compound is an anticonvulsant and the second compound is a psychotherapeutic agent.

### RELATED APPLICATION INFORMATION

This application claims priority to U.S. Provisional Patent Application Ser. No. 60/759,116, filed Jan. 12, 2006, which is hereby incorporated by reference in its entirety.

### BACKGROUND OF THE INVENTION

1. Field of the Invention

The present invention is in the field of pharmaceutical compositions and methods for reversing weight gain.

2. Description of the Related Art

Obesity is a disorder characterized by the accumulation of excess fat in the body. Obesity has been recognized as one of the leading causes of disease and is emerging as a global problem. Increased instances of complications such as hypertension, non-insulin dependent diabetes mellitus, arteriosclerosis, dyslipidemia, certain forms of cancer, sleep apnea, and osteoarthritis have been related to increased instances of obesity in the general population.

Obesity has been defined in terms of body mass index (BMI). BMI is calculated as weight (kg)/[height (m)]². According to the guidelines of the U.S. Centers for Disease Control and Prevention (CDC), and the World Health Organization (WHO) (World Health Organization. Physical status: The use and interpretation of anthropometry. Geneva, Switzerland: World Health Organization 1995. WHO Technical Report Series 854), for adults over 20 years old, BMI falls into one of these categories: below 18.5 is considered underweight, 18.5-24.9 is considered normal, 25.0-29.9 is considered overweight, and 30.0 and above is considered obese.

Certain medications are characterized by the adverse effect of weight gain. Specifically, many antidepressants and antipsychotics are known to cause weight gain. For example, the commonly-prescribed antidepressants Prozac, Zoloft, and Paxil are associated with weight gain. Newer generation antidepressants seem less likely to be associated with cardiovascular side effects and toxicity associated with older generation antidepressants, such as tricyclic antidepressants or monoamine oxidase inhibitors (MAOIs). Currently, newer generation antidepressants include selective serotonin reuptake inhibitors (e.g., fluoxetine, fluvoxamine, sertraline, paroxetine, citalopram, and escitalopram), venlafaxine, duloxetine, nefazodone, mianserin setiptiline, viqualine trazodone, cianopramine, and mirtazapine. Weight gain has been a major concern with certain of the newer antidepressants, particularly, with paroxetine (PAXIL® PAXIL CR®) and mirtazapine (Fava, J. Clin. Psych. 61 (suppl. 11):37-41 (2000); Carpenter et al, J. Clin. Psych. 60:45-49 (1999); Aronne et al, J. Clin. Psych. 64 (suppl. 8):22-29 (2003), both of which are incorporated by reference herein in their entirety).

A large proportion of patients treated with paroxetine, mirtazapine, and other antidepressants, such as venlafaxine (EFFEXOR®, EFFEXOR XR®), gain a significant amount of weight. Most of these patients find it difficult to lose the weight gained as a result of treatment, even after discontinuing use of the particular antidepressant. Importantly, many compounds effective in preventing weight gain are less effective or ineffective in reversing weight gain. The physiology regulating food intake and energy expenditure can differ between obese and non-obese animals (Lin et al., International Journal of Obesity 24:639-646 (2000); El-Haschimi et al., J of Clinical Investigation 105:1827-1832 (2000), both of which are incorporated by reference herein in the their entireties). For example, high fat diet-induced obesity in mice were shown to exhibit insensitivity to leptin. Leptin is a hormone with corresponding receptors in the hypothalamus. In control animals, leptin can regulate fat mass. Though leptin treatment can inhibit feeding and increase energy expenditure in control animals, obese animals are insensitive to leptin, thereby suppressing this effective.

Weight gain associated with medications can cause the weight-related complications described above and can be drastic enough to cause obesity. Additionally, weight gain is unacceptable in patients and a major reason for noncompliance with antidepressant therapy (Cash et al, Percep. Motor Skills 90:453-456 (2000); Deshmukh et al, Cleveland Clinic J. Med. 70:614-618 (2003), both of which are incorporated by reference herein in their entireties). Therefore, medications most effective in treating a psychiatric disorder are frequently either not prescribed by the physician or not regularly taken by the patient.

Individuals would benefit from the availability of a composition that effectively reverses weight gain caused by the administration of a psychotherapeutic medication.

### SUMMARY OF THE INVENTION

In some embodiments, the present invention relates to a method of at least partially reversing weight gain, including identifying an individual who experienced or who is experiencing weight gain, and administering to the individual a composition comprising a therapeutically effective amount of a first and a second compound, wherein the first compound comprises an anticonvulsant and the second compound comprises a psychotherapeutic agent, and wherein the composition does not contain a therapeutically effective amount of topiramate. The first compound may be selected from the group consisting of zonisamide, bupropion, valproic acid, quetiapine, clonazepam, and pharmaceutically acceptable salts or prodrugs thereof. In some embodiments, the first compound is zonisamide. In other embodiments, the first compound is a combination of bupropion and zonisamide. The second compound may be selected from the group consisting of mirtazapine, setiptiline, paroxetine, venlafaxine, olanzapine, bupropion, risperidone, lamotrogine, risperidone, a lithium salt, valproic acid, and pharmaceutically acceptable salts or prodrugs thereof. In some embodiments, the second compound is olanzapine. In other embodiments, the second compound is mirtazapine. In still other embodiments, the second compound is septitiline. The second compound may be a combination of olanzapine and valproate. In some embodiments, the first compound is zonisamide and the second compound is olanzapine. In other embodiments, the first compound is a combination of zonisamide and bupropion and the second compound is olanzapine.

In some embodiments, the first compound can ameliorate the non-weight-related adverse side effects of the second compound. The first compound may be administered at substantially the same time as the second compound, or the first compound may be administered either before or after the second compound. In some embodiments, the first compound and the second compound are combined in a single dosage form. The first compound and the second compound may be administered to the subject in an amount that is effective to synergistically treat weight gain.

In some embodiments, the individual is obese or is characterized by a body mass index greater than 25. In some embodiments, the weight gain was at least partially caused by administration of a psychotherapeutic agent. In some embodiments, the weight gain was at least partially caused by administration of an antidepressant. In some embodiments, the weight gain was at least partially caused by administration of an anticonvulsant.

In some embodiments, the method further includes suppressing the individual's appetite by administration of the composition. In some embodiments, the method further includes increasing the individual's energy expenditure by administration of said composition. In some embodiments, the method further includes treating a weight-related condition that is a metabolic risk factor associated with the weight gain by administration of said composition. The metabolic risk factor may be selected from hypertension, diabetes, arteriosclerosis, dyslipidemia, cancer, sleep apnea, and osteoarthritis.

In some embodiments, the method may further include wherein the body mass index of the individual is decreased by at least 1.0 unit. The individual can, in some embodiments, be in need of a psychotherapeutic agent. In other embodiments, the individual is not in need of a psychotherapeutic agent.

In some embodiments, the present invention relates to a method of treating a weight-related side effect, including identifying an individual who experienced weight gain or who is experiencing associated with the administration of a psychotherapeutic agent, and administering to the individual a composition comprising an anticonvulsant, wherein the composition does not contain a therapeutically effective amount of topiramate, and wherein the administration of the composition at least partially reverses weight gain associated with the administration of the psychotherapeutic agent. The anticonvulsant may be selected from the group consisting of zonisamide, bupropion, valproic acid, quetiapine, clonazepam, and pharmaceutically acceptable salts or prodrugs thereof. In some embodiments, the anticonvulsant is zonisamide. In other embodiments, the anticonvulsant is a combination of bupropion and zonisamide. The psychotherapeutic agent may be selected from the group consisting of mirtazapine, setiptiline, paroxetine, venlafaxine, olanzapine, bupropion, risperidone, lamotrogine, risperidone, a lithium salt, valproic acid, and pharmaceutically acceptable salts or prodrugs thereof. In some embodiments, the psychotherapeutic agent is olanzapine. In some embodiments, the anticonvulsant is zonisamide and the psychotherapeutic agent is olanzapine. In other embodiments, the anticonvulsant is a combination of zonisamide and bupropion and the psychotherapeutic agent is olanzapine.

In some embodiments, the present invention relates to a package including a first compound in unit dosage form and a second compound in unit dosage form and written instructions advising the reader to administer said compounds to the intended recipient to treat weight gain or a weight-related condition, wherein the first compound is an anticonvulsant and the second compound is a psychotherapeutic agent, and wherein the said package does not contain a therapeutically effective amount of topiramate. In some embodiments, the first compound and the second compound are combined in a single unit dosage form. The first compound may be selected from the group consisting of zonisamide, bupropion, valproic acid, quetiapine, clonazepam, and pharmaceutically acceptable salts or prodrugs thereof. In some embodiments, the first compound is zonisamide. In other embodiments, the first compound is a combination of bupropion and zonisamide. The second compound may be selected from the group consisting of mirtazapine, setiptiline, paroxetine, venlafaxine, olanzapine, bupropion, risperidone, lamotrogine, risperidone, a lithium salt, valproic acid, and pharmaceutically acceptable salts or prodrugs thereof. In some embodiments, the second compound is olanzapine. In other embodiments, the second compound is mirtazapine. In still other embodiments, the second compound is septitiline. The second compound may be a combination of olanzapine and valproate. In some embodiments, the first compound is zonisamide and the second compound is olanzapine. In other embodiments, the first compound is a combination of zonisamide and bupropion and the second compound is olanzapine.

These and other embodiments are described in greater detail below.

### BRIEF DESCRIPTION OF THE DRAWINGS

FIG. 1 is a graph showing the effect of zonisamide (10 μM) on the frequency of action currents in POMC neurons. Zonisamide reversibly increased the activity of POMC neurons.

FIG. 2 is a graph showing the effect of olanzapine (100 nM) on the rate of action currents in POMC neurons. Olanzapine reversibly decreased the activity of POMC neurons.

FIG. 3 is a graph showing zonisamide reversed the inhibition of POMC neurons caused by olanzapine cotreatment.

### DETAILED DESCRIPTION OF THE PREFERRED EMBODIMENT

Without being bound by any particular theory, it is believed that potential mechanisms for the observed weight gain caused by psychotherapeutic drugs include histamine H1 receptor antagonism for mirtazapine, and anticholinergic effects in the case of paroxetine.

Zonisamide is a marketed anticonvulsant indicated as adjunctive therapy for adults with partial onset seizures. Without being bound by any particular theory, it is believed that the mechanism of antiepileptic activity appears to be: 1) sodium-channel blocking; and 2) reduction of inward T-type calcium currents. In addition, zonisamide binds to the GABA/benzodiazepine receptor complex without producing change in chloride flux. Further, zonisamide facilitates serotonergic and dopaminergic neurotransmission and possesses a weak inhibitory effect on carbonic anhydrase. Zonisamide further increases the levels of the glutamate transport protein in the brain.

Zonisamide has been shown to cause significant weight loss (comparable to marketed weight loss medications) in patients presenting with primary obesity (Gadde et al, JAMA 289:1820-1825 (2003), incorporated by reference herein in its entirety). It has been postulated that it is the effect of zonisamide on the CNS concentration of serotonin, dopamine and carbonic anhydrase that is responsible for this effect. There is evidence that zonisamide increases serotonin and dopamine synthesis rates (Hashiguti et al, J Neural Transm Gen Sect. 1993; 93:213-223; Okada et al, Epilepsy Res. 1992; 13:113-119, both of which are incorporated by reference herein in their entirety). There is further evidence suggesting that zonisamide stimulates dopamine D₂ receptors (Okada et al, Epilepsy Res. 1995; 22:193-205, incorporated by reference herein in its entirety). Zonisamide was well tolerated, fatigue being the only side effect that occurred more frequently than with placebo treatment.

The present inventors have determined that the use of anticonvulsants, such as zonisamide, in general is effective in reversing weight gain, particularly weight gain associated with the use of psychotherapeutic medications such as antidepressants, particularly newer generation of antidepressants, antihistamines, and serotonin receptor antagonists, such as 5HT_{2C} receptor antagonists.

Vocabulary

Throughout the present disclosure, when a particular compound is mentioned by name, for example, zonisamide, bupropion, setiptiline, mirtazapine, or valproate, it is understood that the scope of the present disclosure encompasses pharmaceutically acceptable salts, esters, amides, or prodrugs of the named compound. Also, if the named compound comprises a chiral center, the scope of the present disclosure also includes compositions comprising the racemic mixture of the two enantiomers, as well as compositions comprising each enantiomer individually substantially free of the other enantiomer. Thus, for example, contemplated herein is a composition comprising the S enantiomer substantially free of the R enantiomer, or a composition comprising the R enantiomer substantially free of the S enantiomer. By “substantially free” it is meant that the composition comprises less than 10%, or less than 8%, or less than 5%, or less than 3%, or less than 1% of the minor enantiomer. If the named compound comprises more than one chiral center, the scope of the present disclosure also includes compositions comprising a mixture of the various diastereomers, as well as compositions comprising each diastereomer substantially free of the other diastereomers. Thus, for example, commercially available mirtazapine is a racemic mixture comprising two separate enantiomers. The recitation of “mirtazapine” throughout this disclosure includes compositions that comprise the racemic mixture of mirtazapine, the compositions that comprise the (+) enantiomer substantially free of the (−) enantiomer, and the compositions that comprise the (−) enantiomer substantially free of the (+) enantiomer.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl) methylamine, and salts thereof with amino acids such as arginine, lysine, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like, or a pbase, as described above.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The term “psychotherapeutic” includes, but is not limited to, antidepressants, anticonvulsants, antipsychotics, and the like.

Anticonvulsants

A variety of compounds and combinations thereof are suitable for use as an anticonvulsant in the methods and compositions described herein. In some embodiments, a composition comprises a first compound that is an anticonvulsant, and in some but not all of these embodiments, the composition can comprise a second compound that is a psychotherapeutic agent. Examples of anticonvulsants include barbiturates, benzodiazepines, GABA analogues, hydantoins, sulfa drugs, miscellaneous anticonvulsants, phenyltriazines, and succinimides. An example of a barbiturate includes pentobarbital. Examples of benzodiazepines include clonazepam, clorazepate, benzodiazepine, and diazepam. Examples of GABA analogues include tiagabine, pregabalin, and gabapentin. Examples of hydantoins include fosphenyloin, phenyloin, and 5,5-Diphenylhydantoin. Examples of miscellaneous anticonvulsants include carbamazepine, valproate, valproic acid, divalproex, felbamate, levetiracetam, carbamazepine, oxcarbazepine, and zonisamide. In some embodiments, the term “anticonvulsant” excludes topiramate. In some embodiments, the anticonvulsant can be a combination of more than one compound. An example of a phenyltriazine is lamotrigine. Examples of succinimides include methsuximide and ethosuximide. Also included are extended release or controlled release formulations of the above drugs, pharmaceutically acceptable salts or prodrugs thereof, as well as combinations of the above drugs. In some embodiments, anticonvulsants only include compounds whose primary purpose is an anticonvulsant.

The anticonvulsant can be a methane-sulfonamide derivatives, such as those described in U.S. Pat. No. 4,172,896, or other sulfamates (including sulfamate-substituted monosaccharides), such as those described in U.S. Pat. No. 4,513,006, incorporated by reference herein in its entirety.

In some embodiments, the anticonvulsant can be bupropion (Tutka et al, Eur J Pharmacol. 2004; 499:117-120).

In some preferred embodiments, the anticonvulsant can be zonisamide. With regard to the pharmacokinetics of zonisamide, its renal excretion and minimal potential for inhibition or induction of hepatic microsomal enzymes, are favorable qualities in the concept of combination use with antidepressants, particularly newer generation antidepressants.

In some embodiments, the anticonvulsant is an anticonvulsant that inhibits carbonic anhydrase. In some embodiments, the anticonvulsant is an anticonvulsant that blocks sodium channels. In some embodiments, the anticonvulsant is an anticonvulsant that reduces inward T-type calcium currents. In some embodiments, the anticonvulsant is an anticonvulsant that inhibits the uptake of GABA and/or enhances the uptake of glutamate. In some embodiments, the anticonvulsant is an anticonvulsant that facilitates serotonergic and/or dopaminergic neurotransmission.

In some embodiments, routine experimentation, informed by the guidance provided herein, may be used to identify an anticonvulsant suitable for use in combination with a particular psychotherapeutic agent in the methods and compositions described herein.

Psychotherapeutic Agent

A variety of compounds and combinations thereof are suitable for use as a psychotherapeutic agent in the methods and compositions described herein. A psychotherapeutic agent may be an antipsychotic, and an antipsychotic may be a psychotherapeutic agent. In some embodiments, a composition is administered to an individual, wherein the composition comprises a first compound, which is an anticonvulsant, and a second compound, which is a psychotherapeutic agent. In other embodiments, a composition is administered to an individual already being administered a psychotherapeutic agent, wherein the composition comprises a first compound that is an anticonvulsant, and optionally a second compound, which is a psychotherapeutic agent, distinct from that already being administered.

In some embodiments the psychotherapeutic agent is an antidepressant, an antimigrane, an antibipolar, an antimania drug, a mood stabilizer, or an antiepileptic. Examples of antidepressants include paroxetine, mirtazapine, and bupropion. Examples of antimigrane drugs include sumatriptan, zolmitriptan, elatriptan and other triptans. Examples of antibipolar drugs include lithium, valproate, carbamezepine, oxycarbamezepine, lamotrogine, tiagabine, olanzapine, clozapine, risperidone, quetiapine, aripiprazole, ziprasidone, and benzodiazepines. In some embodiments, the psychotherapeutic agent comprises a salt of lithium. In other embodiments, the psychotherapeutic agent is valproate, which includes both the salt of valproate and the free acid form of valproic acid. Also included are pharmaceutically acceptable salts or prodrugs of these drugs, extended release or controlled release formulations of the above drugs, as well as combinations of the above drugs. In some embodiments, the lithium salt may be lithium carbonate or lithium citrate. In some embodiments, the lithium drug is in an extended release formulation.

In certain embodiments, the psychotherapeutic agent is a compound of Formula I

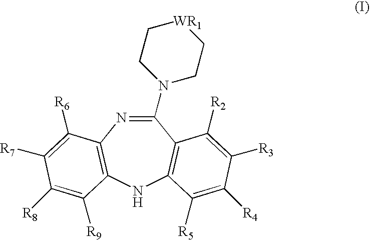

where
W is nitrogen, CH, oxygen, or sulfur;
R₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, optionally substituted C₁₋₆ alkoxyalkyl, and optionally substituted aryl and arylalkyl;
R₂, R₃, R₄, and R₅, are each independently selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, heteroalkyl, and NO₂;
R₆, R₇, R₈, and R₉, are each independently selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, heteroalkyl, and NO₂.

In another embodiment, the psychotherapeutic agent is a tricyclic antidepressant. Examples of tricyclic antidepressants include, but are not limited to, imipramine, desipramine, trimipramine, nortriptyline, clomipramine, doxepin, amitriptyline, maprotiline, protriptyline, dothiapen, setiptiline, cianopramine, and maprotiline. Maprotiline, a very effective antidepressant, is not used widely because it carries risk of seizures. The combination of maprotiline and zonisamide or other anticonvulsants has the added benefit of reducing the risk of seizures associated with the use of maprotiline, in addition to reducing the risk of weight gain due to the use of the antidepressant. The same is also true for combining zonisamide with clomipramine, another tricyclic associated with a relatively higher risk of seizures.

In further embodiments, the psychotherapeutic agent is a monoamine oxidase inhibitor (MAO inhibitor). Examples of MAO inhibitors include, but are not limited to, phenelzine (Nardil®), tranylcypromine (Parnate®), isocarboxazid (Marplan®), moclobemide (Aurorix®), brofaromine, cimoxatone, clorgyline, and lazabemide.

In certain embodiments, the psychotherapeutic agent is an antihistamine, such as setiptilinie, teciptiline, ORG 8282 (Organon, Netherlands), or MO 8282 (Mochida, Japan).

In some embodiments, the psychotherapeutic agent is a 5HT_{2C} receptor antagonist, such as colzapine, N-desmethylclozapine, or clozapine-N-oxide.

In further embodiments, the psychotherapeutic agent a compound disclosed in U.S. Pat. Nos. 3,819,706 and 3,885,046, both of which are incorporated by reference herein in their entirety, are used.

In additional embodiments, the psychotherapeutic agent is a compound that enhances the activity of norepinephrine and/or dopamine, such as by reuptake inhibition or other mechanism. Such compounds can include norepinephrine agonists, such as phendimetrazine and benzphetamine; norepinephrine reuptake inhibitors such as atomoxetine, bupropion, radafaxine, thionisoxetine, and reboxetine; dopamine agonists, such as cabergoline, amantadine, lisuride, pergolide, ropinirole, pramipexole, and bromocriptine; norepinephrine releasers, for example diethylpropion; a mixed dopamine/norepinephrine reuptake inhibitor, for example, bupropion; a combination of a dopamine reuptake inhibitor and a norepinephrine reuptake inhibitor, e.g. bupropion and mazindol; or a combination of a selective serotonin reuptake inhibitor (SSRI) and a norepinephrine reuptake inhibitor, such as sibutramine, venlafaxine, and duloxetine.

In some embodiments, the psychotherapeutic agent can be a combination of more than one compound, such as mirtazapine and bupropion or setiptiline and bupropion.

Routine experimentation, informed by the guidance provided herein, may be used to identify a psychotherapeutic agent suitable for use in combination with a particular anticonvulsant in the methods and compositions described herein.

Compound Combinations

In some embodiments, a composition of the present invention can include a combination of the following compounds:

the first compound is zonisamide and the second compound is mirtazapine;

the first compound is zonisamide and the second compound is setiptiline;

the first compound is zonisamide and the second compound is paroxetine;

the first compound is zonisamide and the second compound is venlafaxine;

the first compound is zonisamide and the second compound is olanzapine;

the first compound is zonisamide and the second compound is bupropion;

the first compound is zonisamide and the second compound is risperidone;

the first compound is zonisamide and the second compound is a salt of lithium;

the first compound is zonisamide and the second compound is valproic acid, or a pharmaceutically acceptable salt, such as different salts of valproate, ester, amide, or prodrugs thereof;

the first compound is zonisamide and the second compound is an agent that enhances the activity of norepinephrine and/or dopamine via uptake inhibition of other mechanism;

the first compound is valproic acid, or a pharmaceutically acceptable salt, such as different salts of valproate, ester, amide, or prodrugs thereof and the second compound is quetiapine;

the first compound is an agent that blocks kainite/AMPA (D,L-α-amino-3-hydroxy-5-methyl-isoxazole propionic acid) subtype glutamate receptors) and the second compound is an agent that enhances the activity of norepinephrine and/or dopamine via uptake inhibition of other mechanism;

the first compound is an agent that blocks kainite/AMPA (D,L-α-amino-3-hydroxy-5-methyl-isoxazole propionic acid) subtype glutamate receptors) and the second compound is bupropion;

the first compound is a combination of zonisamide and clonazepam and the second compound is lamotrogine;

the first compound is zonisamide and the second compound is a combination of mirtazapine and risperidone;

the first compound is zonisamide and the second compound is a combination of paroxetine and bupropion; and

the first compound is zonisamide and the second compound is a combination of setiptiline and bupropion.

Although typically bupropion is considered a psychotherapeutic agent, bupropion may have anticonvulsant properties and can therefore, in some embodiments, be the first compound. In some embodiments, a composition of the present invention can include a combination of the following compounds:

the first compound is bupropion and the second compound is mirtazapine;

the first compound is bupropion and the second compound is setiptiline;

the first compound is bupropion and the second compound is paroxetine;

the first compound is bupropion and the second compound is venlafaxine;

the first compound is bupropion and the second compound is olanzapine;

the first compound is bupropion and the second compound is risperidone;

the first compound is bupropion and the second compound is a salt of lithium;

the first compound is bupropion and the second compound is valproic acid, or a pharmaceutically acceptable salt, such as different salts of valproate, ester, amide, or prodrugs thereof;

the first compound is bupropion and the second compound is an agent that enhances the activity of norepinephrine and/or dopamine via uptake inhibition of other mechanism;

the first compound is a combination of zonisamide and bupropion and the second compound is mirtazapine;

the first compound is a combination of zonisamide and bupropion and the second compound is olanzapine;

the first compound is combination of zonisamide and bupropion and the second compound is setiptiline; and

the first compound is combination of zonisamide and bupropion and the second compound is a combination of olanzapine and valproic acid, or a pharmaceutically acceptable salt, such as different salts of valproate, ester, amide, or prodrugs thereof.

In other embodiments, methods can include identifying an individual already being administered a psychotherapeutic agent, which can be an antidepressant, and administering to the individual a composition comprising zonisamide or another anticonvulsant, wherein the administered composition can be sufficient to reverse or inhibit a weight-related condition associated with the psychotherapeutic agent. The weight-related condition may be inhibited by reversing weight gain associated with the administration of the psychotherapeutic agent. In some of these embodiments, the composition does not include a psychotherapeutic agent, although in other embodiments, the composition does include a psychotherapeutic agent. When the composition does include a psychotherapeutic agent, the psychotherapeutic agent of the composition can be different from the psychotherapeutic agent that was already being administered to the individual.

In some embodiments, when an individual is already receiving an antidepressant, methods described herein can comprise administering to the individual a composition comprising an anticonvulsant and, optionally, also comprising a psychotherapeutic drug, as described herein, wherein the psychotherapeutic drug is not the antidepressant. In some of these embodiments, the psychotherapeutic drug is not an antidepressant. For example, an individual who is taking mirtazapine or setiptiline can be administered a composition comprising zonisamide, a composition comprising valproate and zonisamide, or a composition comprising venlafaxine and zonisamide.

The combination of, for example, zonisamide and bupropion (including sustained release or controlled release preparations) provides an effective means of minimizing metabolic risks associated with weight gain and/or antidepressant use (e.g., type II diabetes). The combination may be more effective than, for example, zonisamide treatment alone and with fewer side effects. Neuropharmacologically, all three major nerve transmitters that regulate appetite and weight, i.e., serotonin, norepinephrine and dopamine, are targeted with the combination of, for example, bupropion and zonisamide. Side effects of, for example, zonisamide (such as somnolence, psychomotor slowing, cognitive impairment, fatigue and depression) may be offset by insomnia, activation, psychomotor agitation and antidepressant effects of, for example, bupropion. On the other hand, zonisamide, for example, may reduce the seizure risk associated with, for example, bupropion. Lower doses of both types of medication can be used in the combination treatment, thereby further reducing the overall side effect burden.

Methods

In some embodiments, the present invention relates to a method of treating a condition, comprising identifying an individual with the condition and administering to the individual a composition described herein. In some of these embodiments, the condition is a weight-related condition. In some embodiments, the weight-related condition is obesity or a condition characterized by a body mass index greater than 25. In other embodiments, the weight-related condition is weight gain. The weight-related condition can be a condition in which it is desirable to suppress the individual's appetite, to increase the individual's satiety, or to increase the individual's energy expenditure. The weight-related condition may be a secondary condition to, for example, a disease.

The treating the weight gain condition may consist of reversing the weight gain. As used herein, reversing may mean partially reversing. In some embodiments, reversal of weight gain can indicate that the patient's weight or BMI returned to the patient's weight or BMI before an antipsychotic treatment was started. In other embodiments, the patient's weight or BMI may return to a healthy level. For example, the patient's BMI may be reduced until the patient is no longer considered obese or overweight, or the patient's weight may be reduced until it considered healthy for the patient's height and sex. In other embodiments, the patient's BMI is reduced, although not always to pre-weight gain levels. The reduction in BMI may be from about 1 to about 50 units. BMI can be reduced by about 1, 2, 3, 4, 5, 6, 7, 8, 9, 10, 11, 12, 13, 14, 15, 20, 25, 30, 35, 40, 45, or 50 units, or a range defined by any of those values.

Compositions used herein may be administered to a patient who experienced weight gain. In some embodiments, the weight gain was experienced during the administration of a psychotherapeutic agent. Such a weight gain may be defined as any increase in the patient's weight or BMI, or alternatively, it may be defined as an increase in the patient's weight or BMI that is larger than would have been expected if the patient was not administered a psychotherapeutic agent.

The weight-related condition may also be a metabolic risk factor associated with weight gain, such as hypertension, diabetes, arteriosclerosis, dyslipidemia, certain forms of cancer, sleep apnea, or osteoarthritis. In some embodiments, the weight-related condition can include a potential weight-related condition. For example, in one embodiment, the method is a method of reducing the risk of developing diabetes after an observed weight gain occurred in an individual who is or was on antidepressant therapy. The patient may have previously been on a psychotherapeutic therapy or may currently be on a psychotherapeutic therapy.

In some preferred embodiments, the weight-related condition is associated with the administration of an antidepressant, an antihistamine, or a serotonin receptor antagonist. In more preferred embodiments, the weight-related condition is associated with the administration of an antidepressant. Some embodiments of the present invention provide methods of reversing weight gain associated with antidepressant therapy. These methods involve the use of weight-loss promoting anticonvulsants, such as zonisamide. In some embodiments, methods of the present invention are also effective against individuals who have gained or who are at risk of gaining weight irrespective of the use of antidepressants. Compositions described herein may reverse weight gain associated with a psychotherapeutic agent or an antidepressant in addition to reversing weight gain not associated with the psychotherapeutic agent or the antidepressant.

In other embodiments, the weight-related condition is associated with the administration of an anticonvulsant, such as, for example, valproate, carbamazepine and gabapentin. Some embodiments of the present invention provide methods of reversing weight gain associated with anticonvulsant therapy. These methods involve the use of weight-loss promoting psychotherapeutic agents. In some embodiments, methods of the present invention are also effective against individuals who have gained or who are at risk of gaining weight irrespective of the use of anticonvulsants.

Administration of a composition disclosed herein to an individual with a condition described above can also reverse weight gain or reverse the increase in the individual's BMI, by reducing the weight gained by the patient or the decreasing the BMI of the patient. In embodiments in which the composition comprises an anticonvulsant, the weight gain or the increase in the individual's BMI can be reversed more than expected by administration of a comparable composition not containing the anticonvulsant. In embodiments in which the composition comprises both an anticonvulsant and a psychotherapeutic agent, the weight gain or the increase in the individual's BMI can also be reversed more than expected by administration of a comparable composition not containing the psychotherapeutic agent.

In some embodiments, administration of a composition disclosed herein can cause the reversal of weight gain in an individual by increasing the individual's physical activity. In other embodiments, administration of a composition disclosed herein can cause the reversal of weight gain in an individual by decreasing the individual's caloric intake. In still other embodiments, administration of a composition disclosed herein can cause the reversal of weight gain in an individual neither by increasing the individual's physical activity nor by decreasing the individual's caloric intake.

In some embodiments, administering a composition disclosed herein to an individual with a weight-related condition described above can result in an inhibition or amelioration of symptoms associated with the weight-related condition.

In some embodiments, an individual is administered a composition comprising both an anticonvulsant and a psychotherapeutic agent. In some embodiments, the anticonvulsant can act as an agent reversing weight gain. In other embodiments, the psychotherapeutic agent can act as an agent reversing weight gain. In still other embodiments, both the anticonvulsant and the psychotherapeutic agent can act as agents reversing weight gain, and they can act synergistically. In one embodiment, either the anticonvulsant or psychotherapeutic agent does not act as an agent reversing weight gain while administered without the other compound while the other compound does act as an agent reversing weight gain while administered without the other compound. The compound that does not act as an agent reversing weight gain while administered without the other compound can, when administered with the other compound, enhance the reversing of weight gain of the other compound.

In some embodiments, the anticonvulsant can act to reverse weight gain. In other embodiments, the psychotherapeutic agent can act to reverse weight gain. In still other embodiments, both the anticonvulsant and the psychotherapeutic agent can act to reverse weight gain, and they can act synergistically.

Patient Identification

Compositions described herein can be administered to an individuals who have experienced weight gain. In some embodiments, the weight gain was at least partially caused by the administration of a psychotherapeutic compound. In some of these embodiments, the psychotherapeutic compound is an antidepressant. The individual may be experiencing or at risk of experiencing a weight-related condition associated with the weight gain. The individual may or may not be continuing to gain weight at the time that the composition is first administered. The individual may be selected from the group consisting of mice; rats; rabbits; guinea pigs; dogs; cats; sheep; goats; cows; primates, such as monkeys, chimpanzees, and apes; and humans.

In certain embodiments, the individual is overweight, characterized by a body mass index (BMI) greater than 25. In other embodiments, the individual is obese, characterized by a BMI greater than 30. In still other embodiments, the individual has a BMI greater than 40. However, in some embodiments, the individual may have a BMI less than 25. In some of these embodiments, it may be beneficial for health or cosmetic purposes to affect weight loss, thereby reducing the BMI even further. The individual may also be underweight, characterized by a BMI less than 18.5. In some embodiments, the individual has reached the above BMI as the result of psychotherapeutic therapy. In other embodiments, the individual has reached the above BMI as the result of anticonvulsant therapy. In still other embodiments, the individual has reached the above BMI independent of either psychotherapeutic or anticonvulsant therapy.

In some embodiments, the individual can be one who is already or was previously being administered a psychotherapeutic therapy. In some of these embodiments, the psychotherapeutic therapy is an antipsychotic therapy. In some embodiments, the psychotherapeutic therapy is an antidepressant therapy. While individuals suitable for treatment in accordance with the instant invention can be receiving or about to begin receiving any antidepressant associated with weight gain, typically, the antidepressant is a newer generation antidepressant (e.g., a selective serotonin uptake inhibitor (e.g., fluoxetine, fluvoxamine, sertraline, paroxetine, citalopram, and escitalopram), venlafaxine, nefazodone, and mirtazapine)), particularly, paroxetine or mirtazapine. In some of these embodiments, the individual can be administered a composition comprising an anticonvulsant, such as zonisamide, or an anticonvulsant and a psychotherapeutic drug, as described herein, wherein the psychotherapeutic drug is not the antidepressant of the antidepressant therapy, and in some embodiments, is not an antidepressant.

In some embodiments, the individual can be one who is already on or who is about to begin anticonvulsant therapy. The anticonvulsant therapy may be associated with either weight loss. In some of these embodiments, the individual can be administered a composition comprising a psychotherapeutic drug or an anticonvulsant and a psychotherapeutic drug, as described herein, wherein the anticonvulsant is not the anticonvulsant of the anticonvulsant therapy.

In some embodiments, the individual is not in need of psychotherapeutic or anticonvulsant therapy.

Administration

In some embodiments, an individual is administered a composition comprising a first compound and a second compound, wherein the first compound is an anticonvulsant and the second compound is a psychotherapeutic agent. The first compound and the second compound may be administered more or less simultaneously. Alternatively, the first compound may be administered prior to the second compound, or the first compound may be administered subsequent to the second compound.

In certain embodiments, the first compound and the second compound are administered individually. However, in other embodiments, the two compounds are joined together by a chemical linkage, such as a covalent bond, so that the two different compounds form separate parts of the same molecule. The chemical linkage is selected such that after entry into the body, the linkage is broken, such as by enzymatic action, acid hydrolysis, base hydrolysis, or the like, and the two separate compounds are then formed.

In embodiments in which the patient is already being administered or about to be administered a psychotherapeutic compound, a composition described herein can be administered more or less simultaneously, before, or after the psychotherapeutic compound. The composition can be administered separately from the psychotherapeutic compound or the composition can be joined by a chemical linkage to the psychotherapeutic compound.

In embodiments in which the patient is already being administered or about to be administered an anticonvulsant compound, a composition described herein can be administered more or less simultaneously, before, or after the anticonvulsant compound. The composition can be administered separately from the anticonvulsant compound or the composition can be joined by a chemical linkage to the anticonvulsant compound.

In any of the embodiments described herein, the first and second compounds can be administered as a single unit dosage form.

Routes of Administration and Formulations

The exact formulation and route of administration for the pharmaceutical compositions described herein can be chosen by the individual physician in view of the patient's condition. (See e.g., Fingl et al. 1975, in “The Pharmacological Basis of Therapeutics”, Ch. 1 p. 1). Details of some embodiments of the appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein, all of which are incorporated herein by reference in their entirety, including any drawings.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally, including sublingually, which include push-fit capsules made of gelatin, as well as soft, sealed capsules made of gelatin and a plasticizer, such as glycerol or sorbitol. The push-fit capsules can contain the active ingredients in a mixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Controlled release forms of the compositions described herein are specifically contemplated, including sustained release formulations. Controlled or sustained release compositions include compositions where one, two or more of the active ingredients in the composition have a controlled or a sustained release profile, while none, one, two, or more other active ingredients have an immediate release profile. Methods for formulating controlled release forms are known to those skilled in the art and may be applied to make controlled release compositions using routine experimentation informed by the guidance provided herein.

The compositions described herein can be included in a food or beverage product. In some preferred embodiments, the compositions can be included in a food or beverage product similar to a craved substance. The food or beverage product can be a beverage, a soup, a solid, a semi-solid, or a frozen confection. The beverage can be a still beverage or a carbonated beverage, and moreover, can be a suspension, for example, a shake, frappe, or float. Carbonated beverages are preferably made without phosphoric acid, to permit a higher pH. Carbonated beverages as diluents are preferably used with buffer formulations of the composition, such that the final pH is greater than about 6. Both carbonated and non-carbonated beverages can be “diet” beverages made with low calorie or no-calorie sweeteners, including saccharine, aspartame, dihydrochalcones, monellin, steviosides, glycyrrhizin, sorbitol, mannitol, maltitol, and others. The beverage can be an infusion or extract, including a tea or a coffee. The solid can be a bar, much like an energy bar or a candy bar; a chip, like a potato or corn chip in shape or texture; a baked good; a non-baked extruded food product; a puffed snack; a cracker; a cookie; in which the solid can be with or without embedded flavor nuggets such as nuts, fruits, or chocolate chips. The semi-solid snack can be a custard, a dessert pudding, a thick cream, a mousse, a parfait, a yogurt, a jelly, a sweetened gelatin, and similar snacks. The frozen confection can be an “ice cream”, an “ice milk”, a sherbet, a flavored ice, and similar snacks, and can, optionally, include a wafer or cone, a stick, cup, or flavor nuggets such as nuts and candy sprinkles (a.k.a. “jimmies”). The frozen confection can be formed into any of a variety of attractive shapes including cones, cups, bars, and sandwiches. The compounds can also be in a powder form. Preferably the powder is free-flowing and readily mixable with water or other fluid. The powder can be mixed with a variety of fluids. Thus, for example, the powder form of the invention can be mixed with water, soda, diet soda, tea, coffee, fruit juice, diet fruit juice, flavored diet beverages, and the like. Preferably, the powder form of the invention is mixed with water or other fluid before drinking. In some embodiments, the compounds can be delivered in the form of a toothpaste.

For buccal administration, the compositions may take the form of tablets or lozenges formulated in conventional manner.

For administration by inhalation, the compounds described herein can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In some embodiments, the invention relates to a pharmaceutical composition comprising a combination of a psychotherapeutic agent and an anticonvulsant, as described above, or comprising a linked molecule, as described herein, and a physiologically acceptable carrier, diluent, or excipient, or a combination thereof.

A pharmaceutical carrier for the hydrophobic compounds of the invention can be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Dosages

The amount of either compound of the composition administered in the pharmaceutical compositions described herein can vary with the patient, other medications that the patient is receiving, the route of administration and the result sought. Optimum dosing regimens for particular patients can be readily determined by one skilled in the art.

Pharmaceutical compositions suitable for use in the present invention include compositions where the active ingredients are contained in an amount effective to achieve its intended purpose. In some embodiments, a therapeutically effective amount means an amount of compound effective to reduce, and in preferred embodiments to substantially reduce the weight of the subject being treated. In other embodiments, a therapeutically effective amount means an amount of compound effective to reverse, and in preferred embodiments to substantially reverse weight gain of the subject. In still other embodiments, a therapeutically effective amount means an amount of compound effective to prevent, alleviate or ameliorate the treated subject's symptoms of a disease related to the subject's weight gain. Determination of a therapeutically effective amount is well within the capability of those skilled in the art, especially in light of the detailed disclosure provided herein.

The exact dosage the pharmaceutical compositions described herein can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 6000 mg of each ingredient, preferably between 1 mg and 5000 mg, e.g. 25 to 5000 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2500 mg and the total daily dosage by parenteral administration will typically be in the range 0.1 to 400 mg. Suitably the compounds will be administered for a period of continuous therapy, for example for a week or more, or for months or years.

In some embodiments, the dosage range for lithium carbonate, for an oral dose, will result in blood levels of lithium being between about 0.5 and about 1.5 meq/l. In a preferred embodiment, the lithium carbonate dosage range, for an oral dose, will be about 900 mg/day.

In certain embodiments, the dosage range for valproate, for an oral dose, is in the range of about 250 to about 5000 mg/day. In a preferred embodiment, the valproate dosage range, for an oral dose, will be about 1500 mg/day.

In further embodiments, the dosage range for zonisamide, for an oral dose, is in the range of about 25 to about 600 mg per day. In some embodiments, the dosage is 25 mg per day. In other embodiments, the dosage is 50 mg per day. In yet other embodiments, the dosage is 100 mg per day.

In further embodiments, the dosage range for mitrazepine, for an oral dose, is in the range of about 5 to about 500 mg per day. In some embodiments, the dosage is 8 mg per day. In other embodiments, the dosage is 16 mg per day. In yet other embodiments, the dosage is 32 mg per day. In some embodiments, the dosage is 15 mg per day. In other embodiments, the dosage is 30 mg per day. In yet other embodiments, the dosage is 45 mg per day.

In other embodiments, the dosage range for venlafaxin or venlafaxin XR, for an oral dose, is in the range of about 20 mg to about 600 mg per day. In some embodiments, the dosage is 25 mg per day. In other embodiments, the dosage is 37.5 mg per day. In yet other embodiments, the dosage is 50 mg per day. In some embodiments, the dosage is 75 mg per day. In other embodiments, the dosage is 100 mg per day. In yet other embodiments, the dosage is 150 mg per day.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The composition can be administered in a controlled-release or extended-release dosage form. The composition can be administered to the patient before, during, or after a specific meal or before, during, or after every meal. The composition can be administered before the patient goes to sleep or in the morning.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

An embodiment provides a package comprising a first compound and a second compound in unit dosage form as described herein, along with instructions advising the reader to administer the unit dosage form to the intended recipient to inhibit a weight-related condition.

It will be understood by those of skill in the art that numerous and various modifications can be made without departing from the spirit of the present invention. Therefore, it should be clearly understood that the forms of the present invention are illustrative only and are not intended to limit the scope of the present invention.

All documents and other information sources cited above are hereby incorporated in their entirety by reference, as are Gadde et al, Obesity Res. 9:544-551 (2001) and Gadde et al, JAMA 289:1820-1825 (2003).

### Some Embodiments of the Invention

Some of the embodiments of the present invention are as follows:

In the first embodiment, the invention relates to a composition for affecting weight loss comprising a first compound and a second compound, wherein said first compound is an anticonvulsant and said second compound is a psychotherapeutic agent.

In the second embodiment, the invention relates to the composition of the first embodiment, wherein said psychotherapeutic agent is selected from the group consisting of lithium carbonate, lithium citrate, valproate, mixtures thereof, and pharmaceutically acceptable salts or prodrugs thereof.

In the third embodiment, the invention relates to the composition of the first embodiment, wherein said anticonvulsant is selected from the group consisting of a barbiturate, benzodiazepine, GABA analogue, hydantoins, anticonvulsant, phenyltriazine, succinimide, pharmaceutically acceptable salts or prodrugs thereof, and combinations thereof.

In the fourth embodiment, the invention relates to the composition of the third embodiment, wherein said barbiturate is pentobarbital or pharmaceutically acceptable salts or prodrugs thereof.

In the fifth embodiment, the invention relates to the composition of the third embodiment, wherein said benzodiazepine is selected from the group consisting of clonazepam, alprazolam, chlordiazepoxide, clorazepate, diazepam, halazepam, lorazepam, oxazepam, prazepam, flurazepam, temazepam, triazolam, pharmaceutically acceptable salts or prodrugs thereof, and combinations thereof.

In the sixth embodiment, the invention relates to the composition of the third embodiment, wherein said GABA analogue is selected from the group consisting of tiagabine, gabapentin, pregabalin, pharmaceutically acceptable salts or prodrugs thereof, and combinations thereof.

In the seventh embodiment, the invention relates to the composition of the third embodiment, wherein said hydantoin is selected from the group consisting of fosphenyloin, phenyloin, 5,5-Diphenylhydantoin, pharmaceutically acceptable salts or prodrugs thereof, and combinations thereof.

In the eighth embodiment, the invention relates to the composition of the third embodiment, wherein said miscellaneous anticonvulsant is selected from the group consisting of carbamazepine, valproate, valproic acid, divalproex, felbamate, levetiracetam, carbamazepine, oxcarbazepine, zonisamide, pharmaceutically acceptable salts or prodrugs thereof, and combinations thereof.

In the ninth embodiment, the invention relates to the composition of the third embodiment, wherein said phenyltriazine is lamotrigine.

In the tenth embodiment, the invention relates to the composition of the third embodiment, wherein said succinimide is selected from the group consisting of methsuximide, ethosuximide, and combinations thereof.

In the eleventh embodiment, the invention relates to the composition of the first embodiment, wherein said first compound is a zonisamide and said second compound is a pyschotherapeutic agent.

In the twelfth embodiment, the invention relates to the composition of the first embodiment, wherein said first compound is zonisamide and said second compound is lithium carbonate or lithium citrate.

In the thirteenth embodiment, the invention relates to the composition of the first embodiment, wherein said first compound is zonisamide and said second compound is valproate.

In the fourteenth embodiment, the invention relates to the composition of the twelfth or thirteenth embodiment, wherein the zonisamide is in a time-release formulation.

In the fifteenth embodiment, the invention relates to a method of reversing weight gain, comprising identifying an individual in need thereof and treating that individual with a psychotherapeutic agent and an anticonvulsant.

In the sixteenth embodiment, the invention relates to the method of the fifteenth embodiment, wherein said individual has a body mass index greater than 25.

In the seventeenth embodiment, the invention relates to the method of the fifteenth embodiment, wherein the psychotherapeutic agent is selected from the group consisting of lithium carbonate, lithium citrate, and valproate, extended release or controlled release formulations of the above drugs, and combinations of the above drugs.

In the eighteenth embodiment, the invention relates to the method of the fifteenth embodiment, wherein the anticonvulsant is selected from the group consisting of barbiturates, benzodiazepines, GABA analogues, hydantoins phenyltriazines, and succinimides, and pharmaceutically acceptable salts or prodrugs thereof.

In the nineteenth embodiment, the invention relates to the method of the fifteenth embodiment, wherein the anticonvulsant is selected from the group consisting of pentobarbital, clonazepam, clorazepate, benzodiazepine, diazepam, tiagabine, gabapentin, pregabalin, fosphenyloin, phenyloin, phenyloin, 5,5-Diphenylhydantoin, carbamazepine, valproate, valproic acid, divalproex, felbamate, levetiracetam, carbamazepine, oxcarbazepine, zonisamide, lamotrigine, methsuximide, ethosuximide, extended release or controlled release formulations of the above drugs, and combinations of the above drugs.

In the twentieth embodiment, the invention relates to the method of the fifteenth embodiment, wherein said first compound and said second compound are administered nearly simultaneously.

In the twenty first embodiment, the invention relates to the method of the fifteenth embodiment, wherein said first compound is administered prior to said second compound.

In the twenty second embodiment, the invention relates to the method of the fifteenth embodiment, wherein said first compound is administered subsequent to said second compound.

In the twenty third embodiment, the invention relates to a method of increasing satiety in an individual comprising identifying an individual in need thereof and treating that individual with a first compound and a second compound, wherein said first compound is an anticonvulsant and said second compound is a psychotherapeutic agent.

In the twenty fourth embodiment, the invention relates to the method of the twenty third embodiment, wherein said first compound and said second compound are administered nearly simultaneously.

In the twenty fifth embodiment, the invention relates to the method of the twenty third embodiment, wherein said first compound is administered prior to said second compound.

In the twenty sixth embodiment, the invention relates to the method of the twenty third embodiment, wherein said first compound is administered subsequent to said second compound.

In the twenty seventh embodiment, the invention relates to a method of increasing energy expenditure in an individual comprising identifying an individual in need thereof and treating that individual with a first compound and a second compound, wherein said first compound is an anticonvulsant and the second compound is a psychotherapeutic agent.

In the twenty eighth embodiment, the invention relates to the method of the twenty seventh embodiment, wherein said first compound and said second compound are administered nearly simultaneously.

In the twenty ninth embodiment, the invention relates to the method of the twenty seventh embodiment, wherein said first compound is administered prior to said second compound.

In the thirtieth embodiment, the invention relates to the method of the twenty seventh embodiment, wherein said first compound is administered subsequent to said second compound.

In the thirty first embodiment, the invention relates to a method of suppressing the appetite of an individual comprising identifying an individual in need thereof and treating that individual with a first compound and a second compound, wherein said first compound is an anticonvulsant and said second compound is a psychotherapeutic agent.

In the thirty second embodiment, the invention relates to the method of the thirty first embodiment, wherein said first compound and said second compound are administered nearly simultaneously.

In the thirty third embodiment, the invention relates to the method of the thirty first embodiment, wherein said first compound is administered prior to said second compound.

In the thirty fourth embodiment, the invention relates to the method of the thirty first embodiment, wherein said first compound is administered subsequent to said second compound.

In the thirty fifth embodiment, the invention relates to a method of reversing weight gain in an individual comprising identifying an individual in need thereof and treating that individual with a combination of lithium carbonate and zonisamide.

In the thirty sixth embodiment, the invention relates to a method of reversing weight gain in an individual comprising identifying an individual in need thereof and treating that individual with a combination of valproate and zonisamide.

In the thirty seventh embodiment, the invention relates to the method of the thirty fifth or thirty sixth embodiments, wherein the individual has a BMI greater than 30.

In the thirty eighth embodiment, the invention relates to the method of the thirty fifth or thirty sixth embodiments, wherein the individual has a BMI greater than 25.

In the thirty ninth embodiment, the invention relates to the method of the thirty fifth or thirty sixth embodiments, wherein the lithium carbonate or valproate is in a time-release formulation.

In the fortieth embodiment, the invention relates to the method of the thirty fifth or thirty sixth embodiments, wherein the plasma concentration level of both the lithium carbonate or valproate and zonisamide follow a similar concentration profile.

In the forty first embodiment, the invention relates to the method of the thirty ninth embodiment, wherein the lithium carbonate or valproate and the zonisamide are administered substantially simultaneously.

In the forty second embodiment, the invention relates to the method of the thirty ninth embodiment, wherein the lithium carbonate or valproate is administered prior to the zonisamide.

In the forty third embodiment, the invention relates to the method of the thirty ninth embodiment, wherein the lithium carbonate or valproate is administered subsequent to the zonisamide.

In the forty fourth embodiment, the invention relates to a method of promoting weight loss in an individual who has experienced weight gain after administration of a psychotherapeutic agent, comprising identifying an individual in need thereof and treating that individual with an anticonvulsant.

In the forty fifth embodiment, the invention relates to the method of the forty fourth embodiment, wherein the psychotherapeutic agent is olanzapine.

In the forty sixth embodiment, the invention relates to the method of the forty fourth embodiment, wherein the anticonvulsant is zonisamide.

In the forty seventh embodiment, the invention relates to the method of the forty fourth embodiment, wherein the psychotherapeutic agent is olanzapine and the anticonvulsant is zonisamide.

### EXAMPLES

The examples below are non-limiting and are merely representative of various aspects of the invention.

### Example 1

### Use of Zonisamide Alone

Individuals who are currently taking or who previously took an antidepressant and who have gained weight as the result of the use of the antidepressant are identified. Each individual is instructed to take one 25 mg tablet of zonisamide on a daily basis, alone or in addition to the antidepressant therapy.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

The dosage of zonisamide can be from about 25 mg to about 800 mg per day, generally given once per day or divided (e.g., equally) into multiple doses. Preferably, the dose is from about 100 mg to about 600 mg per day, more preferably, the dose is from about 200 mg to about 400 mg per day. However, it may be necessary to use dosages outside these ranges. Zonisamide tablets are usually made and marketed in 25 mg, 50 mg, and 100 mg doses. Individual tablets, or combination of tablets can be used to achieve the desired dosing. If the initial dosages are not effective, they can be increased.

### Example 2

### Combination of Zonisamide and Mitrazepine

Individuals having a BMI of greater than 25 are identified. Each individual is instructed to take one tablet of zonisamide on a daily basis, in addition to one tablet of mitrazepine on a daily basis. Initially, the drugs are administered as follows: 8 mg mitrazepine and 64 mg zonisamide; or 16 mg mitrazepine and 128 mg zonisamide; or 32 mg mitrazepine and 252 mg zonisamide; generally with an mitrazepine/zonisamide ratio of 1:8.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

If the initial dosages are not effective, they can be increased.

### Example 3

### Combination of Zonisamide and Paroxetine

Individuals having a BMI of greater than 25 are identified. Each individual is instructed to take one tablet of zonisamide on a daily basis, in addition to one tablet of paroxetine on a daily basis. Initially, the drugs are administered as follows: 10 mg paroxetine and 60 mg zonisamide; or 20 mg paroxetine and 120 mg zonisamide; or 30 mg paroxetine and 180 mg zonisamide; or 40 mg paroxetine and 240 mg zonisamide; generally with an paroxetine/zonisamide ratio of 1:6.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

If the initial dosages are not effective, they can be increased.

### Example 5

### Combination of Zonisamide and Lithium Carbonate

Individuals having a BMI of greater than 25 are identified. Each individual is instructed to take one 25 mg tablet of zonisamide on a daily basis, in addition to one 300 mg tablet of lithium carbonate on a daily basis.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs. It is recommended that serum creatinine be checked periodically.

If the initial dosage is not effective, then the zonisamide dosage can be increased by approximately 25 mg per day or the lithium carbonate dosage can be increased so as to achieve blood levels of 0.5 to 1.5 meq/l. If the initial dosage results in a more rapid weight loss than the above rate, the dosage of each of zonisamide or lithium carbonate can be reduced.

In some cases, it is beneficial to administer one dose of zonisamide per day in conjunction with two or three or more doses of lithium carbonate throughout the day. Lithium carbonate may also be in a time-release formulation where the dose is administered once a day, but lithium carbonate gradually enters the blood stream throughout the day, or in the course of a 12 hour period.

The above procedure can be followed using lithium citrate, or any other pharmaceutically acceptable salt of lithium, instead of lithium carbonate.

### Example 6

### Combination of Zonisamide and Valproate

Individuals having a BMI of greater than 25 are identified. Each individual is instructed to take one 50 mg tablet of zonisamide on a daily basis, in addition to one 500 mg tablet of valproate on a daily basis.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

If the initial dosage is not effective, then the zonisamide dosage can be increased by approximately 30 mg per day, though not exceeding 600 mg total per day. If the initial dosage results in a more rapid weight loss than the above rate, the dosage of each of zonisamide or valproate can be reduced.

In some cases, it is beneficial to administer one dose of zonisamide per day in conjunction with two or three or more doses of valproate throughout the day. Valproate may also be in a time-release formulation where the dose is administered once a day, but valproate gradually enters the blood stream throughout the day, or in the course of a 12 hour period.

### Example 7

### Combination of Zonisamide and Valproate

Individuals having a BMI of greater than 25 are identified. Each individual is instructed to take one 50 mg tablet of zonisamide on a daily basis. In addition, each individual is instructed to take one 250 mg tablet of valproate on a daily basis.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

If the initial dosage is not effective, then the valproate dosage can be increased by 20 mg intervals up to 3000 mg per day. If the initial dosage results in a more rapid weight loss than the above rate, the dosage of each of zonisamide or valproate can be reduced.

### Example 8

### Combination of Zonisamide and Olanzapine

Individuals having a BMI of greater than 25 are identified. Each individual is instructed to take one tablet of zonisamide on a daily basis, in addition to one tablet of olanzapine on a daily basis. Initially, the drugs are administered as follows: 5 mg olanzapine and 60 mg zonisamide, or 10 mg olanzapine and 120 mg zonisamide; generally with an olanzapine/zonisamide ratio of 1:12.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

If the initial dosages are not effective, they can be increased.

### Example 9

### Combination of Zonisamide and Risperidone

Individuals having a BMI of greater than 25 are identified. Each individual is instructed to take one tablet of zonisamide on a daily basis, in addition tablet of risperidone on a daily basis. Initially, the drugs are administered as follows: 0.5 mg risperidone and 30 mg zonisamide, 1 mg risperidone and 60 mg zonisamide, or 2 mg risperidone and 120 mg zonisamide; generally with an olanzapine/zonisamide ratio of 1:60.

The individuals are monitored for a period of time, preferably months. It is recommended that the dosage be adjusted so that each individual loses weight at a rate of 10% of initial weight every 6 months. However, the rate of weight loss for each individual may be adjusted by the treating physician based on the individual's particular needs.

If the initial dosages are not effective, they can be increased.

### Example 10

### Zonisamide Prevents the Weight Gain Associated with Olanzapine Treatment

Background

In this example, the effect of co-treatment with drug combinations on the weight gain associated with olanzapine use was tested in rats. Specifically, addition of zonisamide to concomitant olanzapine treatment is shown to decrease the weight gain associated with olanzapine, in a validated rodent model of olanzapine-induced weight gain.

The melanocortin system consists of Proopiomelanocortin (POMC) neurons, the cognate melanocortin receptors (MC4 R) and the agouti-related peptide neurons in the arcuate nucleus of the hypothalamus. It is well established in humans and animals that the melanocortin system controls energy balance, and the most common genetic cause of obesity in humans is congenital lack of MC4 R. It has recently been shown that many compounds that influence energy balance modify the activity of melanocortin circuits. In particular, it has been shown that increases the electrophysiological activity of POMC neurons. It has also been shown that cannabinoid antagonists activate this same circuitry. Some of the receptors that can regulate the activity of POMC neurons have been identified; in particular it has been shown that 5-HT 2C and 5-HT 1B receptors increase the activity of POMC neurons as does dopamine D2 R. The clear role of D2 R and 5-HT 2CR in regulating the activity of POMC neurons shows that compounds like olanzapine, which is an antagonist at both these receptors, modify energy balance to induce an anabolic state, favoring weight gain.

In Vivo Pharmacology

Female Sprague-Dawley rats, weighing about 235 grams at the start of the experiment were used. They were trained to sham injections, using the zonisamide vehicle for 2 weeks before the study commenced. Under isoflurane anesthesia, Alzet osmotic minipumps (2 ml2) were implanted subcutaneously, between the shoulder blades. The rats were subsequently returned to their home cages after recovery. The minipumps delivered 5 μL per hour for 14 days. Olanzapine was dissolved in 1.5% lactic acid in dH₂O. Zonisamide was dissolved in 10% DMSO, 13.4% EtOH, 20.1% PPG, and 66.5% saline. Olanzapine dose was 1.75 mg/day via the minipump. The animals were housed individually and supplied with standard laboratory chow. Food consumed and animal weights were recorded every days. There were 5 animals in the control (vehicle) group, 5 animals in the zonisamide only group, 4 animals in the olanzapine only group, and 6 animals in the olanzapine+zonisamide group.

Rats were allowed to recover after pump implantation, and then received twice daily injections of zonisamide 26 mg/kg. Their food intake and body weight were calculated daily for 15 days.

The results of these studies are shown below:

| | | | | | | | Olanzapine + | |
|---|---|---|---|---|---|---|---|---|
| | Vehicle | | Zonisamide | | Olanzapine | | Zonisamide | |
| Day | Mean | SEM | Mean | SEM | Mean | SEM | Mean | SEM |
| Cumulative Change in Body Weight | | | | | | | | |
| 1.00 | 6.10 | 2.64 | 9.74 | 2.98 | 7.44 | 3.74 | 3.33 | 2.35 |
| 4.00 | 19.24 | 5.16 | 20.24 | 2.94 | 21.04 | 3.56 | 12.13 | 4.36 |
| 5.00 | 17.14 | 6.39 | 17.60 | 2.97 | 19.22 | 5.15 | 12.67 | 3.57 |
| 6.00 | 19.76 | 6.49 | 19.34 | 4.05 | 20.56 | 6.18 | 12.05 | 2.65 |
| 7.00 | 23.96 | 5.78 | 20.18 | 2.62 | 30.88 | 4.83 | 15.67 | 3.96 |
| 8.00 | 22.80 | 5.81 | 17.32 | 2.97 | 27.56 | 6.28 | 10.40 | 3.68 |
| 9.00 | 25.80 | 4.44 | 13.66 | 3.40 | 32.94 | 7.83 | 20.50 | 3.05 |
| 10.00 | 24.78 | 7.24 | 14.62 | 6.02 | 34.98 | 6.35 | 16.45 | 3.28 |
| 11.00 | 26.60 | 6.59 | 16.46 | 3.60 | 33.40 | 6.46 | 18.55 | 2.44 |
| 12.00 | 27.14 | 6.46 | 21.08 | 3.27 | 40.76 | 7.91 | 18.58 | 2.43 |
| 13.00 | 29.68 | 6.35 | 24.26 | 3.56 | 38.84 | 9.07 | 24.83 | 2.31 |
| 14.00 | 34.04 | 6.33 | 27.52 | 3.63 | 43.28 | 8.60 | 21.65 | 2.60 |
| 15.00 | 28.70 | 5.61 | 25.70 | 6.26 | 41.64 | 9.34 | 22.22 | 3.70 |
| Change in Food Intake Post | | | | | | | | |
| Pump Implant | | | | | | | | |
| 1.00 | 15.84 | 1.68 | 12.48 | 1.79 | 16.56 | 1.29 | 15.17 | 0.68 |
| 2.00 | 32.34 | 1.96 | 30.10 | 2.99 | 39.82 | 1.93 | 35.22 | 1.22 |
| 5.00 | 52.72 | 2.65 | 46.02 | 4.13 | 62.30 | 4.36 | 52.60 | 1.35 |
| 6.00 | 71.82 | 2.68 | 58.24 | 3.16 | 86.14 | 6.02 | 72.82 | 1.74 |
| 7.00 | 87.88 | 4.27 | 72.10 | 3.32 | 105.74 | 7.17 | 89.98 | 3.63 |
| 8.00 | 101.70 | 5.52 | 86.88 | 3.28 | 119.30 | 7.12 | 107.47 | 2.58 |
| 9.00 | 121.84 | 5.99 | 104.86 | 3.57 | 144.98 | 8.35 | 125.95 | 2.16 |
| 10.00 | 139.82 | 6.03 | 123.66 | 3.31 | 161.16 | 9.37 | 144.15 | 3.22 |
| 11.00 | 157.54 | 5.33 | 140.40 | 3.10 | 180.96 | 10.62 | 158.10 | 4.73 |
| 12.00 | 175.35 | 4.59 | 160.36 | 2.10 | 201.42 | 10.92 | 177.27 | 4.81 |
| 13.00 | 195.70 | 3.66 | 177.70 | 5.53 | 224.38 | 11.57 | 197.35 | 6.06 |
| 14.00 | 217.93 | 3.84 | 186.25 | 10.97 | 241.48 | 12.45 | 219.13 | 5.98 |

Electrophysiology

The electrophysiological activity of Proopiomelanocortin (POMC) neurons in brain slices from POMC-EGFP mice was recorded. The POMC neurons in these mice are identified by the expression of green fluorescent protein (EGFP) in these, and only these, cells. The frequency of action potentials in these neurons were recorded using standard electrophysiological techniques. In particular, loose cell attached patch configuration is used to determine action current frequency, whilst minimally disturbing the cells.

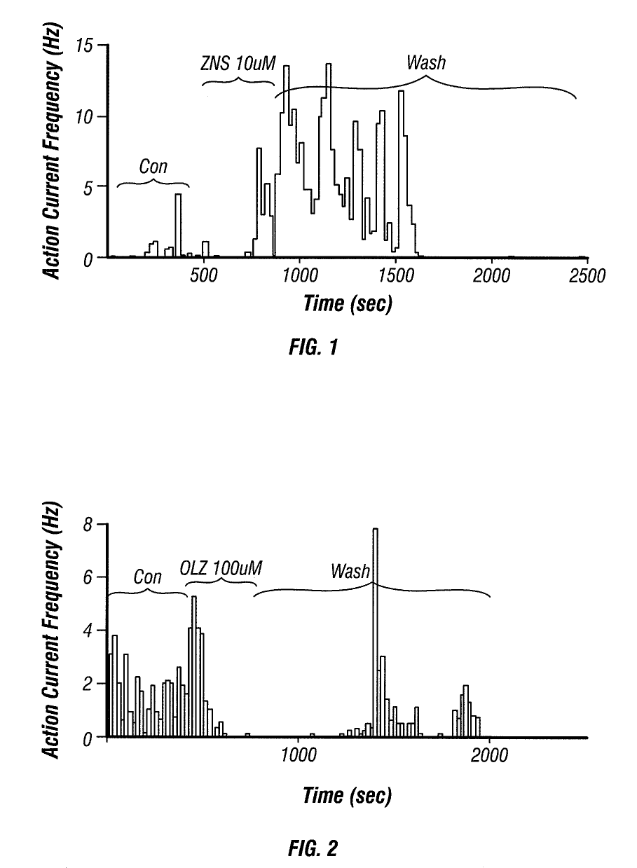

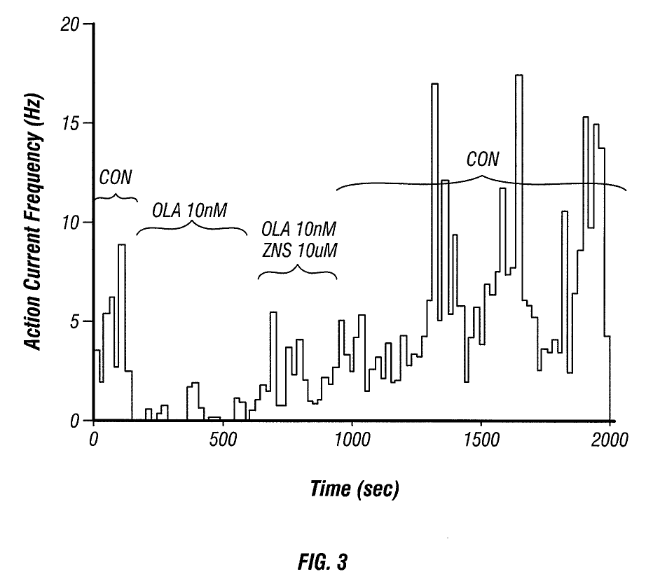

It has been shown that zonisamide increases the activity of POMC neurons, as shown in FIG. 1. The basal activity was recorded, and then olanzapine (100 nM) was added to the tissue bath to determine the effect of olanzapine on the activity of POMC neurons. Olanzapine reduced the activity of POMC neurons, as shown in FIG. 2. Pharmacological data show that olanzapine decreases the activity of POMC neurons. Then the effect of zonisamide on the reduction in activity caused by zonisamide was tested by adding olanzapine plus zonisamide (10 μM) to the bath. An increase in neuronal activity was observed, as shown in FIG. 3.

### Example 11

### Zonisamide Reverses the Weight Gain Associated with Olanzapine Treatment

In this experiment, the effect of zonisamide treatment on the weight gain associated with olanzapine use is tested. Specifically, treatment with zonisamide one week after implantation of olanzipine pumps is shown to decrease the weight gain associated with olanzapine, in a validated rodent model of olanzapine-induced weight gain. The protocol used was the same as described in Example 12, with the exception that the treatment with zonisamide began one week after implantation of the olanzapine osmotic pumps, and food intake and body weight were calculated daily for 10 days.

The results of these studies are shown below:

| | | | | | | | Olanzapine + | |
|---|---|---|---|---|---|---|---|---|
| | Vehicle | | Zonisamide | | Olanzapine | | Zonisamide | |
| Day | Mean | SEM | Mean | SEM | Mean | SEM | Mean | SEM |
| Cumulative Change in Body | | | | | | | | |
| Weiglt Post-Pump Implant | | | | | | | | |
| 7.00 | −3.78 | 1.94 | −1.73 | 1.12 | −0.46 | 0.83 | −2.2 | 1.57 |
| 8.00 | −14 | 1.54 | −11.38 | 1.58 | −11.55 | 1.07 | −11.1 | 3.02 |
| 9.00 | −3.31 | 4.13 | −1.28 | 0.70 | 4.28 | 2.05 | 6.5 | 2.00 |
| 10.00 | −1.4 | 7.47 | 5.8 | 1.50 | 11.88 | 2.87 | 14.16 | 3.05 |
| 11.00 | 0.46 | 4.50 | 6.37 | 2.08 | 13.48 | 3.45 | 15.44 | 3.38 |
| 12.00 | 0.4 | 4.31 | 0.86 | 2.60 | 11.8 | 12.43 | 9.59 | 4.26 |
| 13.00 | −0.68 | 3.03 | −4.79 | 3.24 | 15.55 | 2.90 | 2.45 | 4.14 |
| 14.00 | −0.54 | 1.92 | −11.79 | 3.19 | 20.4 | n = 2 | 0.03 | 3.67 |
| 15.00 | −0.26 | 1.16 | −10.3 | 1.98 | 17.98 | n = 2 | −1.74 | 4.00 |
| 16.00 | 1.88 | 1.79 | −9.37 | 2.51 | 21.22 | n = 2 | −3.48 | 4.22 |
| Cumulative Change in Food | | | | | | | | |
| Intake Post-Pump Implant | | | | | | | | |
| 7.00 | 10.5 | 1.34 | 13.15 | 1.20 | 13.39 | 1.16 | 12.51 | 1.23 |
| 8.00 | 14.59 | 1.77 | 18.62 | 1.25 | 18.36 | 1.18 | 22.51 | 1.68 |
| 9.00 | 43.11 | 5.10 | 46.87 | 1.77 | 57.46 | 1.35 | 61.78 | 2.30 |
| 10.00 | 95.96 | 10.43 | 101.08 | 2.24 | 124.58 | 1.50 | 127.71 | 3.63 |
| 11.00 | 114.35 | 10.55 | 116.93 | 2.97 | 145.5 | 2.54 | 148.27 | 4.09 |
| 12.00 | 130.49 | 10.81 | 126.64 | 3.72 | 163.6 | 2.11 | 156.84 | 4.85 |
| 13.00 | 145.65 | 10.50 | 133.95 | 4.63 | 181.05 | 4.55 | 165.13 | 4.52 |
| 14.00 | 160.76 | 10.18 | 141.09 | 4.88 | 200.9 | n = 2 | 176.71 | 5.21 |
| 15.00 | 176.32 | 9.65 | 153.61 | 4.92 | 218.2 | n = 2 | 189.42 | 5.88 |
| 16.00 | 194.4 | 9.77 | 167.93 | 4.95 | 236.95 | n = 2 | 200.78 | 5.48 |

Thus, zonisamide reversed olanzapine-induced weight gain as evidenced by the reduction of body weight and food intake post-pump implant.

### Example 12

### Case Study of Combination of Zonisamide, Lamotrogine, and Clonazepam

A 49 year-old woman having biopolar disorder, Type I, had been treated for just over year by her psychiatrist using clonazepam 1 mg qHS, fluoxetine 20 mg qd, and lamotrogine 300 mg qD. One particular complaint was that she had “constant thoughts of eating”. Her psychiatrist started her on zonisamide 100 mg qD. She reported that the zonisamide significantly reduced her craving for food and prevented weight gain. The dose was continued and despite multiple stressors at home, she continued to do well on the treatment regimen.

### Example 13

### Case Study of Combination of Zonisamide, Paroxetine, and Risperidone

A 45 year old female patient with social phobia and schizoaffective disorder was treated with paroxetine augmented with risperidone. She had marked increase in appetite and gained 40 pounds. A trial of bupropion alone did not cause significant weight loss. She was therefore started on zonisamide 100 mg, increased to 200 mg. In 3 weeks she lost 12 pounds. The dose was then increased to 300 mg and then to 600 mg. In 5 months her weight returned to baseline. The psychiatric symptoms also improved.

### Example 14

### Case Study of Combination of Zonisamide, Olanzapine, Valproate, and Bupropion

A 30 year-old female patient with a diagnosis of bipolar disorder gained 56 lbs while receiving olanzapine and valproate over 5 years. Despite significant weight gain, as her mental illness was effectively controlled, these medications were continued. She was clinically obese with a BMI of 33.8 kg/m². In an effort to assist in weight reduction, bupropion was added at 150 mg/day while olanzapine and valproate were continued. When bupropion dose was raised to 300 mg/day, the patient reported feeling hyperactive; hence, the dose was reduced back to 150 mg/day. After 6 months, the patient lost 23.6 lbs. However, while continuing to receive bupropion, the patient regained 10.6 lbs over the next 10 months. At this point, zonisamide was added to her medication regimen at 100 mg/day and the dose was increased to 200 mg/day after two weeks. The patient lost 15 lbs over the next 4 months and reported no adverse effects. She remained free of bipolar disorder symptoms.

The case illustrates two advantages of the combination therapy of bupropion and zonisamide. 1) Although bupropion helped in decreasing weight gain resulting from olanzapine and valproate, the patient could not take a higher dose (300-400 mg/day) of bupropion because of precipitation of hypomanic symptoms. The risk of induction of manic or hypomanic symptoms with use of antidepressant medications in susceptible patients is well documented. 2) Zonisamide helped in further offsetting weight gain associated with olanzapine and valproate after the patient lost weight initially with bupropion and regained some of the weight lost.

What is claimed is:

1. A method of at least partially reversing weight gain, comprising:
identifying an individual who experienced or who is experiencing weight gain; and
administering to the individual a composition comprising a therapeutically effective amount of a first and a second compound,
wherein the first compound comprises an anticonvulsant and the second compound comprises a psychotherapeutic agent, and
wherein the composition does not contain a therapeutically effective amount of topiramate.

2. The method of claim 1, wherein the first compound is selected from the group consisting of zonisamide, bupropion, valproic acid, quetiapine, clonazepam, and pharmaceutically acceptable salts or prodrugs thereof.

3. The method of claim 1, wherein the first compound is zonisamide.

4. The method of claim 1, wherein the first compound is a combination of bupropion and zonisamide.

5. The method of claim 1, wherein the second compound is selected from the group consisting of mirtazapine, setiptiline, paroxetine, venlafaxine, olanzapine, bupropion, risperidone, lamotrogine, risperidone, a lithium salt, valproic acid, and pharmaceutically acceptable salts or prodrugs thereof.

6. The method of claim 1, wherein the second compound is olanzapine.

7. The method of claim 1, wherein the second compound is mirtazapine.

8. The method of claim 1, wherein the second compound is setiptiline.

9. The method of claim 1, wherein the second compound is a combination of olanzapine and valproate.

10. The method of claim 1, wherein the first compound is zonisamide and the second compound is olanzapine.

11. The method of claim 1, wherein the first compound is a combination of zonisamide and bupropion and the second compound is olanzapine.

12. The method of claim 1, wherein the first compound ameliorated the non-weight-related adverse side effects of the second compound.

13. The method of claim 1, wherein the first compound is administered at substantially the same time as the second compound.

14. The method of claim 1, wherein the first compound is administered either before or after the second compound.

15. The method of claim 1, wherein the first compound and the second compound are combined in a single dosage form.

16. The method of claim 1, wherein the first compound and the second compound are administered to the subject in an amount that is effective to synergistically treat weight gain.

17. The method of claim 1, wherein the individual is obese or is characterized by a body mass index greater than 25.

18. The method of claim 1, wherein weight gain was at least partially caused by administration of a psychotherapeutic agent.

19. The method of claim 1, wherein weight gain was at least partially caused by administration of an antidepressant.

20. The method of claim 1, wherein weight gain was at least partially caused by administration of an anticonvulsant.

21. The method of claim 1, further comprising suppressing the individual's appetite by administration of said composition.

22. The method of claim 1, further comprising increasing the individual's energy expenditure by administration of said composition.

23. The method of claim 1, further comprising treating a weight-related condition that is a metabolic risk factor associated with the weight gain by administration of said composition.

24. The method of claim 23, wherein the metabolic risk factor is selected from hypertension, diabetes, arteriosclerosis, dyslipidemia, cancer, sleep apnea, and osteoarthritis.

25. The method of claim 1, further comprising wherein the body mass index of the individual is decreased by at least 1.0 unit.

26. The method of claim 1, wherein the individual is in need of a psychotherapeutic agent.

27. The method of claim 1, wherein the individual is not in need of a psychotherapeutic agent.

28. A method of treating a weight-related side effect, comprising:
identifying an individual who experienced weight gain or who is experiencing associated with the administration of a psychotherapeutic agent; and
administering to the individual a composition comprising an anticonvulsant,
wherein the composition does not contain a therapeutically effective amount of topiramate, and
wherein the administration of the composition at least partially reverses weight gain associated with the administration of the psychotherapeutic agent.

29. The method of claim 28, wherein the anticonvulsant is selected from the group consisting of zonisamide, bupropion, valproic acid, quetiapine, clonazepam, and pharmaceutically acceptable salts or prodrugs thereof

30. The method of claim 28, wherein the anticonvulsant is zonisamide.

31. The method of claim 28, wherein the anticonvulsant is a combination of bupropion and zonisamide.

32. The method of claim 28, wherein the psychotherapeutic agent is selected from the group consisting of mirtazapine, setiptiline, paroxetine, venlafaxine, olanzapine, bupropion, risperidone, lamotrogine, risperidone, a lithium salt, valproic acid, and pharmaceutically acceptable salts or prodrugs thereof.

33. The method of claim 28, wherein the psychotherapeutic agent is an antidepressant.

34. The method of claim 28, wherein the psychotherapeutic agent is olanzapine.

35. The method of claim 28, wherein the anticonvulsant is zonisamide and the psychotherapeutic agent is olanzapine.

36. The method of claim 28, wherein the anticonvulsant is a combination of bupropion and zonisamide and the psychotherapeutic agent is olanzapine.

37. A package comprising:
a first compound in unit dosage form and a second compound in unit dosage form; and
written instructions advising the reader to administer said compounds to the intended recipient to treat weight gain or a weight-related condition;
wherein the first compound is an anticonvulsant and the second compound is a psychotherapeutic agent,
and wherein the said package does not contain a therapeutically effective amount of topiramate.

38. The package of claim 37, wherein the first compound and the second compound are combined in a single unit dosage form.

39. The package of claim 37, wherein the first compound is selected from the group consisting of zonisamide, bupropion, valproic acid, quetiapine, clonazepam, and pharmaceutically acceptable salts or prodrugs thereof

40. The package of claim 37, wherein the first compound is zonisamide.

41. The package of claim 37, wherein the first compound is a combination of bupropion and zonisamide.

42. The package of claim 37, wherein the second compound is selected from the group consisting of mirtazapine, setiptiline, paroxetine, venlafaxine, olanzapine, bupropion, risperidone, lamotrogine, risperidone, a lithium salt, valproic acid, and pharmaceutically acceptable salts or prodrugs thereof.

43. The package of claim 37, wherein the second compound is olanzapine.

44. The package of claim 37, wherein the second compound is mirtazapine.

45. The package of claim 37, wherein the second compound is setiptiline.

46. The package of claim 37, wherein the first compound is zonisamide and the second compound is olanzapine.

47. The package of claim 37, wherein the first compound is a combination of bupropion and zonisamide and the second compound is olanzapine.
